(19) United States
(12) Patent Application Publication
(10) Pub. No.: US 2021/0407642 A1
(43) Pub. Date: Dec. 30, 2021

(54) DRUG RECOMMENDATION METHOD AND DEVICE, ELECTRONIC APPARATUS, AND STORAGE MEDIUM

(71) Applicant: Beijing Baidu Netcom Science and Technology Co., Ltd., Beijing (CN)

(72) Inventors: Yuan Xia, Beijing (CN); Zhenhui Shi, Beijing (CN); Chao Lu, Beijing (CN); Haifeng Huang, Beijing (CN)

(73) Assignee: Beijing Baidu Netcom Science and Technology Co., Ltd., Beijing (CN)

(21) Appl. No.: 17/108,367

(22) Filed: Dec. 1, 2020

(30) Foreign Application Priority Data
Jun. 24, 2020 (CN) 202010589770.X

(51) Int. Cl. G16H 20/10; G16H 70/40; G16H 10/60; G06F 40/205

(52) U.S. Cl. CPC G16H 20/10; G06F 40/205; G16H 10/60; G16H 70/40

(57) ABSTRACT

A drug recommendation method and device, an electronic apparatus, and a storage medium are provided, which are related to the fields of artificial intelligence deep learning technology, intelligent recommendation, and knowledge graph. The specific implementation includes: acquiring related information of a target object; and determining drug recommendation information for the target object based on the related information of the target object and a first model, where the drug recommendation information contains information of at least one drug, where the first model is a model obtained by performing iterative processing on output information of a second model, and the second model is used for evaluating drug recommendation information output by the first model during the iterative processing, to obtain an evaluation result of the drug recommendation information.

### CROSS-REFERENCE TO RELATED APPLICATIONS

This application claims priority to Chinese patent application, No. 202010589770.X, entitled “Drug Recommendation Method and Device, Electronic Apparatus, and Storage Medium”, filed with the Chinese Patent Office on Jun. 24, 2020, which is hereby incorporated by reference in its entirety.

### TECHNICAL FIELD

The present application relates to a field of computer technology, and in particular, to fields of artificial intelligence deep learning technology, intelligent recommendation, and knowledge graph.

### BACKGROUND

In existing technology, in the era of information society, every traditional field has been impacted by emerging technologies. The technologies of machine learning and artificial intelligence have achieved milestone breakthroughs in various fields. Nowadays, with the advent of the era of big data and artificial intelligence, more and more large companies and research institutions have begun to enter the fields of Internet medical and intelligent drug recommendation.

### SUMMARY

A drug recommendation method and device, an electronic apparatus, and a storage medium are provided in the present application.

According to a first aspect of the present application, a drug recommendation method is provided. The method includes:

acquiring related information of a target object; and

determining drug recommendation information for the target object based on the related information of the target object and a first model, wherein the drug recommendation information contains information of at least one drug,

wherein the first model is a model obtained by performing iterative processing on output information of a second model, and the second model is used for evaluating drug recommendation information output by the first model during the iterative processing, to obtain an evaluation result of the drug recommendation information.

According to a second aspect of the present application, a drug recommendation device is provided. The device includes:

an information acquisition module, configured to acquire related information of a target object; and

a drug recommendation module, configured to determine drug recommendation information for the target object based on the related information of the target object and a first model, wherein the drug recommendation information contains information of at least one drug,

wherein the first model is a model obtained by performing iterative processing on output information of a second model, and the second model is used for evaluating drug recommendation information output by the first model during the iterative processing, to obtain an evaluation result of the drug recommendation information.

According to a third aspect of the present application, an electronic apparatus is provided. The electronic apparatus includes:

at least one processor; and

a memory communicatively connected to the at least one processor, wherein

the memory stores instructions executable by the at least one processor, the instructions are executed by the at least one processor to enable the at least one processor to perform the aforementioned method.

According to a fourth aspect of the present application, a non-transitory computer-readable storage medium for storing computer instructions is provided. The computer instructions, when executed by a computer, cause the computer to perform the aforementioned method.

By applying embodiments of the present application, a drug recommendation is provided by a first model according to related information of a target object. In addition, the first model is obtained by performing iterative processing on output information of a second model, and the role of the second model is to evaluate drug recommendation information. In this way, since the evaluation of drug recommendation information is introduced in training of the first model, so that finally recommended drugs may be more accurate.

It should be understood that the content described herein is not intended to denote key or critical elements of embodiments of the present application nor to limit the scope of the present application. Further features of the present application may be readily understood from the following description.

### BRIEF DESCRIPTION OF THE DRAWINGS

The drawings are used to better understand the scheme and do not constitute a limitation to the present application, wherein:

FIG. 1 is a schematic flowchart showing a drug recommendation method according to an embodiment of the present application;

FIG. 2 is a schematic diagram showing a processing architecture according to an embodiment of the present application;

FIG. 3 is a schematic flowchart showing model pre-training according to an embodiment of the present application;

FIG. 4 is a schematic diagram showing another processing architecture according to an embodiment of the present application;

FIG. 5 is a schematic flowchart showing model iteration processing according to an embodiment of the present application;

FIG. 6 is a first schematic diagram showing a composition structure of a drug recommendation device according to an embodiment of the present application;

FIG. 7 is a second schematic diagram showing a composition structure of a drug recommendation device according to an embodiment of the present application; and

FIG. 8 is a block diagram showing an electronic apparatus for implementing a drug recommendation method according to an embodiment of the present application.

### DETAILED DESCRIPTION

The exemplary embodiments of the application will be described below in combination with drawings, including various details of the embodiments of the present application to facilitate understanding, which should be considered as exemplary only. Therefore, those of ordinary skill in the art should realize that various changes and modifications can be made to the embodiments described herein without departing from the scope and spirit of the present application. Likewise, descriptions of well-known functions and structures are omitted in the following description for clarity and conciseness.

With the breakthrough of AlphaGo in the field of Go, more and more fields adopt models and methods based on the reinforcement learning. Especially, in consideration of the field of human-computer interaction, the reinforcement learning has its natural advantages. Regarding the medical intelligent drug recommendation, on the one hand, when providing a drug recommendation, effects of the symptomatic treatment and the etiological treatment of recommended drugs should be taken into account, and on the other hand, the incompatibility and interaction between drugs are necessarily to be taken into account, while a reinforcement learning model may enable a system to learn optimal drug recommendation logic and accurately provide a drug recommendation result. Therefore, in the present application, both the accuracy and rationality of a drug recommendation combination are taken into consideration, and how to provide intelligent drug combination recommendation is transformed into an enhancement of model training, to optimize a model. The present application proposes an intelligent drug recommendation method according to data of medical orders and medical records of a target object (e.g., a patient or user), as well as data from medical literatures, national pharmacopoeia, and massive drug instructions.

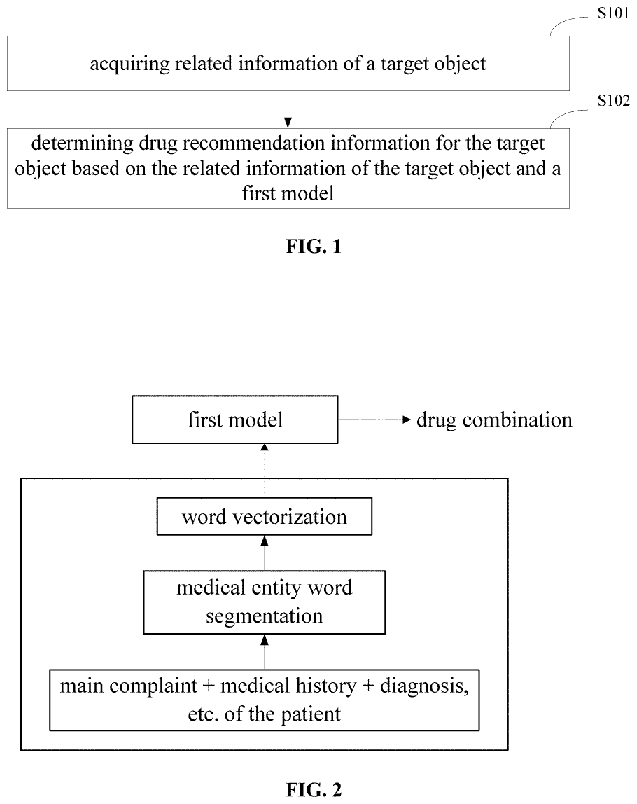

Specifically, a drug recommendation method is provided according to an embodiment of the present application. As shown in FIG. 1, the method includes:

S101: acquiring related information of a target object;

S102: determining drug recommendation information for the target object based on the related information of the target object and a first model, wherein the drug recommendation information contains information of at least one drug,

wherein the first model is a model obtained by performing iterative processing on output information of a second model, and the second model is used for evaluating drug recommendation information output by the first model during the iterative processing, to obtain an evaluation result of the drug recommendation information.

The above scheme relates to the fields of artificial intelligence deep learning technology, intelligent recommendation, and knowledge graph. In addition, the above scheme of the embodiment may be applied to an electronic apparatus, such as a terminal device, a personal computer (PC), a notebook computer, and so on. The above scheme may also be applied to a server, and if so, the acquired related information of the target object may be related information of the target object received from the terminal device. In addition, after being obtained by processing, the final drug recommendation information may be stored, and the drug recommendation information may further be pushed to a terminal device for displaying thereby.

During a determination of drug recommendation using a trained first model, in S101, the target object may be any user or patient. The related information of the target object may be at least one of symptom information, complaint information, diagnosis result information, and medical record information of a user, i.e., the target object.

Correspondingly, in S102, determining drug recommendation information for the target object based on the related information of the target object and a first model includes:

performing word segmentation processing on the related information of the target object, to obtain related information after the word segmentation processing;

performing vectorization processing on the related information after the word segmentation processing, to obtain vectorized related information; and

determining the drug recommendation information for the target object based on the vectorized related information and the first model.

Specifically, the word segmentation processing may be performed on the related information of the target object using a medical entity database. Being different from a word segmentation for a traditional text, since in the present application, a text is for the medical field, a dedicated medical entity database is required to be established first, and then a sentence segmentation is performed based on the medical entity database, to achieve a medical entity word segmentation, so that a sentence may be segmented into respective words, while medical-related words will be marked with respective categories, e.g., “vomiting” will be marked as “symptom”, and “pneumonia” will be marked as “disease”. The word segmentation processing here may be understood as filtering from a large number of input words, to obtain information such as symptom related to a target object (user or patient).

Vectorization processing is performed on the related information obtained after the word segmentation processing, to obtain vectorized related information, that is, after the word segmentation, a word vectorization technology (Word2Vec, GloVe) is adopted, to map words into vector expressions. Finally, the related information of a patient, such as a symptom, is expressed as Vemr (e1, e2, . . . , en), where ei denotes a vectorized representation of a medical entity.

An exemplary description of the above processing is provided in conjunction with FIG. 2. Acquiring related information of a target object refers to acquiring at least one of a main complaint, a medical record, a diagnosis, etc. of any patient who currently needs to acquire a drug recommendation. A medical entity word segmentation processing is performed on the related information of the patient, to obtain related information of the patient after a word segmentation processing. A word vectorization processing is performed based on the related information of the patient after the word segmentation processing, to obtain vectorized information of the medical record of the patient. The vectorized information of the medical record of the patient is input into a first model, so that drug recommendation information, i.e., a recommended drug combination, for the target object, i.e., a patient is determined.

It is also pointed out in S102 that the first model is a model obtained by performing iterative processing on output information of a second model. The second model is used for evaluating drug recommendation information output by the first model during the iterative processing, to obtain an evaluation result of the drug recommendation information. That is, in embodiments of the present application, Actor-Critic reinforcement learning model is used to perform the model iterative processing, and the first model finally obtained may output an optimal drug combination, in which no incompatibility exists between drugs.

The iterative processing mainly includes two parts, i.e., Actor and Critic, where Actor is used for implementing a drug recommendation strategy, and Critic is used for evaluating a rationality of the medication strategy.

The first part relates to an Actor model based on reinforcement learning, i.e., the first model. A drug recommendation is performed by training a deep semantic ranking model as the first model.

The second part relates to a Critic model based on reinforcement learning, i.e., the second model. The Critic model in the reinforcement learning network, i.e., the second model is obtained by training another deep network model in conjunction with knowledge graphs. The second model is used to evaluate a drug combination recommended by a current Actor, to obtain an evaluation result (that is, if there is a negative relationship or incompatibility between drugs, the Critic will give a negative reward).

The first model may be the Actor model based on reinforcement learning. In addition, the first model (or referred to as the Actor model) may be constructed based on a deep semantic ranking network BERT model, and it is a model for ranking the drug recommendations. The BERT-based deep learning method is widely used in various fields of natural language processing, including automatic text classification, sentiment analysis and machine translation, etc., which will not be described in detail here.

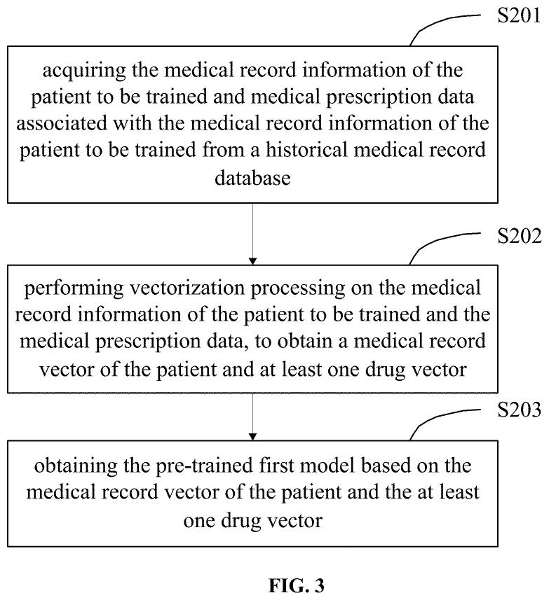

In an example, a pre-training procedure of the first model, as shown in FIG. 3, may include:

S201: acquiring the medical record information of the patient to be trained and medical prescription data associated with the medical record information of the patient to be trained from a historical medical record database, wherein the medical prescription data contains data of at least one drug;

S202: performing vectorization processing on the medical record information of the patient to be trained and the medical prescription data, to obtain a medical record vector of the patient and at least one drug vector;

S203: obtaining the pre-trained first model based on the medical record vector of the patient and the at least one drug vector.

In the pre-training or construction of the first model, learning is made according to a large amount of data of medical orders and medical records based on a deep semantic ranking model (BERT). In the training process, a ranking binary model is constructed based on Electronic Medical Record (EMR) information of the patient and medically ordered drugs (i.e., the first model is constructed or trained).

In S201, a historical medical record database may be understood as a sample database, and the medical record information of the patient to be trained may be EMR information of any patient in the historical medical record database. It should be understood that there may be one or more medical record information of the patient to be trained in the historical medical record database. In the procedure of pre-training the first model, different medical record information of the patient to be trained may be extracted multiple times for each training processing, until the position of the pre-trained first model is obtained. Since the processing procedure of pre-training the first model according to different medical record information of the patient to be trained is the same each time, the description thereto will not be repeated.

In S202, the performing vectorization processing on the medical record information of the patient to be trained and the medical prescription data, to obtain a medical record vector of the patient and at least one drug vector may specifically include:

performing word segmentation processing on the medical record information of the patient to be trained, to obtain medical-related segmented words of the patient; and then performing vectorization processing based on the medical-related segmented words of the patient after the word segmentation processing, to obtain the medical record vector of the patient;

performing vectorization processing on the medical prescription data to be trained, to obtain at least one drug vector,

wherein the performing vectorization processing on the medical prescription data to be trained may further include: performing word segmentation processing on the medical prescription data, to obtain medical prescription segmented words; and then performing vectorization processing on the medical prescription segmented words.

It should be pointed out that the above vectorization processing on the medical prescription data may be performed at the same time as or after the vectorization processing is performed based on the medical-related segmented words of the patient after the word segmentation processing. Related types of word segmentation are executed after vectorization processing. Of course, the vectorization processing may also be performed before the vectorization processing is performed based on the medical-related segmented words after the word segmentation processing, as long as at least one drug vector may be obtained, and the processing sequences is not limited in the procedure.

Specifically, vectorization processing is firstly performed on content of the EMR information of a patient to be trained (which may include main complaint+current medical history+diagnosis, etc.). Unlike a word segmentation of a traditional text, in the present application, here is for a text in the medical field, so a dedicated medical entity database is established at first, and then a sentence segmentation is performed based on the medical entity database, to obtain medical entity segmented words. Thus, a sentence may be segmented into respective words, while medical-related words may be marked with respective categories. For example, “vomiting” will be marked as “symptom” and “pneumonia” will be marked as “disease”. The word segmentation processing here may be understood as filtering from a large number of input words, to obtain medical record information of a patient to be trained, which is related to the symptom and other information of the patient to be trained.

After a word segmentation, words are mapped into expression of vectors using a word vectorization technology (Word2Vec, GloVe). Finally, the patient's EMR is expressed as Vemr (e1, e2, . . . , en), where ei denotes a vectorized representation of a medical entity. In the same way, that is, by using the word vectorization processing, medical order prescription data corresponding to the EMR is parsed into vectors of a plurality of drugs (e.g., aspirin, ribavirin, etc.), i.e., Vdrug (d1, d2, . . . dn), where di represents a vector expression of a drug.

In S203, regarding the construction of the first model, a deep learning framework may be used to construct a pairwise-based BERT deep semantic ranking model.

As compared with a traditional neural network (DNN, RNN) framework, on the one hand, the BERT-based deep network model considers an order relationship between words in a sentence, which is more in line with the basic assumptions of natural language processing (the word order influences the semantic expression), and on the other hand, the BERT is implemented internally based on the Transformer structure, with a self-attention mechanism, which considers a relationship between patient entity word information. Therefore, in a preferred example provided by embodiments of the present application, the BERT model is used to construct the first model. However, in actual processing, it is not excluded that other models may also be used to construct the first model of the present application, which are not exhaustively repeated here.

Further, the BERT model (i.e., the first model or so called Actor model) is based on patient's EMR vectors and corresponding medical order prescription vectors as inputs, and outputs a priority of a single combination, and a medical combination finally takes the drugs of topk.

In the construction of the first model, for a patient i, it may be set that the positive sample is the patient information state Spos=<Viemr, dk∈Vidrug>, and the information state of the negative sample is Sneg=<Viemr, dk∉Vidrug>. In addition, the reward of the positive sample is reward=+ra, and the reward of the negative samples is reward=−ra.

The negative sample is constructed, based on the patient's EMR, by randomly acquiring drugs not in the medical order of the EMR. The rewards of the positive and negative sample may be understood as labels of the positive and negative samples, and may be preset according to actual conditions. The value of ra may also be preset according to actual conditions, such as 1.

After the Actor model has been pre-trained, a strategy Q (Semr, Adrug) according to the patient's recommended drug combinations may be obtained, and an optimal Q* (Semr, Adrug) may be obtained on a training set, where the method of selecting an optimal drug combination may be based on a policy gradient, which will not be described in detail here.

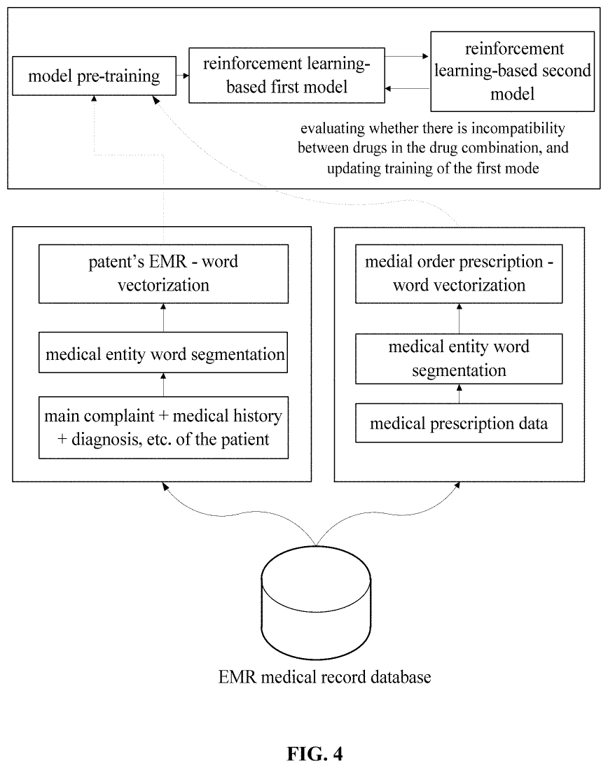

In an example, the construction or pre-training of the first model is described with reference to FIG. 4. Based on a historical medical record database, i.e., an EMR medical record database in FIG. 4, medical record information of a certain patient to be trained and corresponding medical prescription data are obtained, where the medical record data of the patient may include at least one of a main complaint, a medical history, a diagnosis and other information of the patient shown in FIG. 4, and the medical prescription data is that shown in FIG. 4.

The medical entity vectorization processing is performed on the medical record data of the patient, to obtain medical record data of the patient after the vectorization processing, i.e., the medical record vector of the patient obtained by performing the word vectorization processing on the patient's EMR. The medical prescription data is processed in the same way, for example, including the vectorization processing based on a medical entity and the word vectorization processing on a medical prescription, and finally at least one drug vector is obtained.

The first model is pre-trained based on the medical record vector of the patient and at least one drug vector. The pre-trained first model is obtained by repeating the above steps. Here, whether the pre-training of the first model is completed may be determined according to a back propagation of a corresponding loss function. The design of the loss function corresponding to the first model is not described in detail here.

After the pre-training of the aforementioned first model, i.e., the Actor model, is completed, iterative processing may be performed on the first model based on a second model, to obtain a finally applicable first model through reinforcement learning.

Based on the pre-trained deep semantic ranking model (the first model or the so called Actor model), when a system acquires a patient's diagnosis and clinical manifestation as well as crowd information, a reinforcement learning-based first model (or Actor module) completes a drug combination recommendation based on the patient's EMR information. At the same time, a reinforcement learning-based second model (or so called Critic model or module) evaluates a current drug combination recommendation, to determine whether the current drug combination have any drug incompatibility or any negative relationship between drugs, and feeds an obtained reward (+rc, −rc) back to the reinforcement learning agent. If the drug combinations recommended by the reinforcement learning-based Actor module are incompatible, the second model (or referred as the Critic model or module) will give a negative reward, to prompt the reinforcement learning agent to update the strategy function. The iteration is performed, until a final drug combination recommendation given by the Actor has no drug incompatibility under the evaluation by the Critic module.

The reinforcement learning agent may be composed of the above first model and the second model.

Experiments have proved that according to the reinforcement learning-based intelligent consultation method, interaction between drugs represented through the reward function during a training procedure may be taken into account, so that the drug incompatibility in a combined drug recommendation is avoided, and the probability of side effects between drug combinations is reduced, while the symptomatic treatment and etiological treatment are ensured, in this way, the drug recommendation effect may better meet a doctors' expectation.

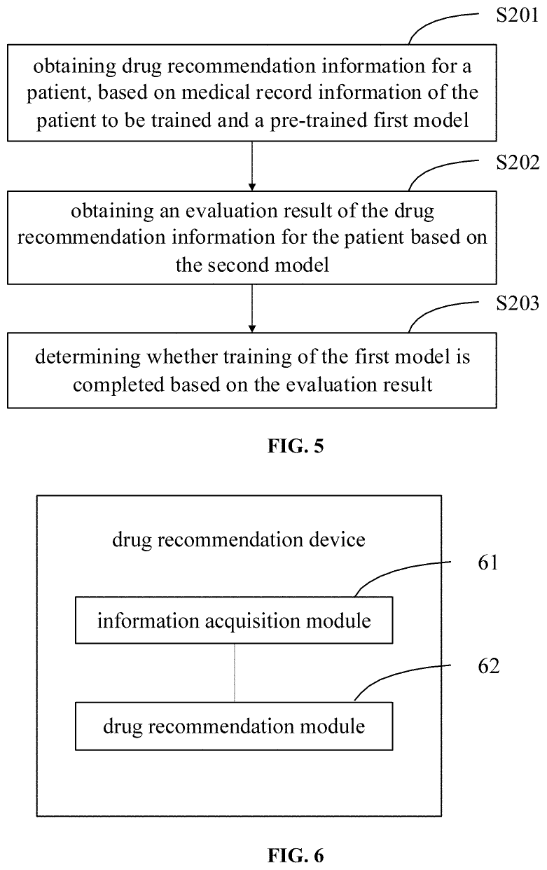

Specifically, in an example, the reinforcement learning of the first model and the second model, i.e., the iterative processing, may be as shown in FIG. 5, including:

S301: obtaining drug recommendation information for a patient, based on medical record information of the patient to be trained and the pre-trained first model;

S302: obtaining an evaluation result of the drug recommendation information for the patient based on the second model, wherein the evaluation result indicates whether there is incompatibility in the drug recommendation information for the patient;

S303: determining whether training of the first model is completed based on the evaluation result.

In S301, in the medical record information of the patient to be trained, only medical record data of the patient is included, such as at least one of a symptom, a main complaint, a medical record, and a diagnosis result of the patient. That is, after a first model is successfully constructed, a second model may be employed to further adjust a weight (or feature parameter) in the first model. At this time, the input of the first model is the medical record data of the patient. The output of the first model is the drug recommendation information for the patient.

In a traditional intelligent drug recommendation method, the rationality of drug combinations (the relationship between drugs and the incompatibility of drugs) are not restricted. Although generated drug combinations may treat symptomatically, there may be drug incompatibility. Therefore, in the present application, a second model, i.e., a reinforcement learning-based Critic module (or model) is provided for further constraints. In S302, it can be understood that a second model (Critic module) is trained to evaluate the drug rationality, i.e., the rationality of a drug combination is scored, to acquire an additional reward (rc).

In S302, obtaining an evaluation result of the drug recommendation information for the patient based on the second model includes:

evaluating a drug combination output by the pre-trained first model based on the second model, to obtain a first reward value corresponding to the drug combination and a probability value of incompatibility between drugs in the drug combination; and taking the first reward value and the probability value as the evaluation result.

Specifically, the second model acquires a knowledge graph G (di, dj) representing drug interactions in conjunction with a rule, to provide a drug score as the aforementioned evaluation result. The second model (Critic module) may determine whether drugs have incompatibility relationships by leaning through a neural network (e.g., DNN) based on relationships constructed by the graph G of drug relationship, which may also be a drug relationship matrix G.

In the drug relationship matrix G, if there is incompatibility between drugs i and j, then G (di, dj)=−a, otherwise G (di, dj)=a, where a is a settable reward value, such as 1; of course, it may also be set to 0.5, 2 and so on according to actual situations, which is not exhaustively repeated here.

In the reinforcement learning training stage, the second model combines the reward value obtained from the evaluation of the drug output of the first model and the probability of the DNN, to give a reward (rc)=G (di, dj)+rp,

where rp is a probability of drug incompatibility determined by the Critic, and where rp=DNN (Adrug).

In S303, determining whether training of the first model is completed based on the evaluation result includes:

updating the training of the first model and updating training of the second model, to obtain an updated-trained first model and an updated-trained second model, in response to determining that the evaluation result indicates there is incompatibility in the drug recommendation information for the patient;

re-obtaining drug recommendation information for the patient by using the updated-trained first model and the medical record information of the patient to be trained, and re-obtaining an evaluation result of the drug recommendation information for the patient based on the updated-trained second model;

determining that the training of the first model is not completed, until the evaluation result indicates that there is no incompatibility in the drug recommendation information for the patient.

In other words, if an evaluation result of the second model indicates that there is incompatibility in the drug recommendation information, the first model needs to be adjusted again, so that the first model may output drug recommendation information having no drug incompatibility, while the second model will also be adjusted accordingly.

It should be understood here that the medical record information of the patient to be trained input into the first model after the updated training and the medical record information of the patient to be trained in S301 may be the same as or different from each other, and they both may be understood as being acquired from the historical medical record database.

In another example, the reinforcement learning of the first model and the second model, i.e., the iterative processing, is also described with reference to FIG. 4. After the pre-training is completed, input of the first model for reinforcement learning may be medical record information of the patient to be trained. Here, the medical record information of the patient to be trained may only include medical record vector of the patient after a word vectorization is performed, i.e., the left branch divided from the EMR medical record database shown in FIG. 4.

Drug recommendation information for the patient is obtained according to the first model and the medical record vector of the patient, the drug recommendation information including a drug combination composed of at least one drug.

An evaluation result of the drug recommendation information for the patient is obtained based on the second model, where the evaluation result indicates whether there is incompatibility in the drug recommendation information for the patient.

The training of the first model is updated, in response to determining that the evaluation result indicates there is incompatibility in the drug recommendation information for the patient. In this case, the method may further include updating the training of the second model based on the updated-trained first model.

The drug recommendation information for the patient is re-obtained by using the updated-trained first model and the medical record information of the patient to be trained, and an evaluation result of the drug recommendation information for the patient is re-obtained based on the updated-trained second model.

The training of the first model is determined being not completed, until the evaluation result indicates that there is no incompatibility in the drug recommendation information for the patient.

Further, the obtaining drug recommendation information for a patient, based on medical record information of the patient to be trained and a pre-trained first model further includes: obtaining a second reward value corresponding to the drug recommendation information for the patient;

correspondingly, the method further includes:

determining a reward function result based on the second reward value and the evaluation result;

training the first model based on the reward function result, until the training of the first model is completed.

Specifically, a final evaluation result (reward (rc)) of the reinforcement learning-based second model and the second reward value (represented as reward (ra)) of the first model are weighted, as a final reward function result (Rf=rc+ra).

In the above processing, through mutual iterations of the reinforcement learning-based Actor module and Critic module and based on the final reward Rf, an optimal Q*F (Semr, Adrug) is generated, thereby providing a result of an optimal intelligent drug combination recommendation.

The method of the present application may be applied to many scenarios, including, but not limited to, clinical decision-making assistance system prescription recommendation, intelligent drug recommendation assistant, medical rational drug use and solution pharmacist prescription teaching, and for teaching interns how to prescribe according to patient's situation, etc.

Being different from a commodity recommendation in the e-commerce field, as to the medical intelligent drug recommendation, not only drugs are required to be recommended according to a diagnosis and clinical manifestation of a known patient, the relationships between drugs and situations of drugs and patients are also required to be taken into account. A series of information such as the main compliant, the medical history, the allergy history, etc. of a patient is required to be comprehensively considered, to provide a most appropriate prescription drug recommendation. This is difficult to be achieved in the whole field of intelligent medical, however, it is important. An idealized intelligent consultation method should not only ensure that recommended drugs may symptomatically treat patient's clinical manifestation, but also be able to etiologically treat according to the diagnosis. In addition, when a drug recommendation is provided, the incompatibility between and the side effects of the drugs are required to still be taken into consideration. However, these are often not easy to be satisfied at the same time.

In the related technology, the medical record mining method based on big data deep learning and the drug recommendation method based on the probabilistic graph of the drug knowledge graph cannot provide satisfactory results. On the one hand, as to a method based on big data deep learning (CNN, LSTM, BERT, etc.), a model may provide symptomatic treatment and etiological treatment when there is enough medical order data in medical records, and medical order data may be completely correct. However, under normal circumstances, it is impossible to acquire a large amount of medical order data, and the quality of medical order data varies from hospital to hospital, while drugs recommended by the medical record mining method based on big data deep learning may be incompatible, which will cause very serious problems in actual use. On the other hand, the method based on the probabilistic graph model (PGM) of the knowledge graph may conduct an effective consultation through a transition probability matrix of drug-indication (diagnosis+clinical manifestation), however, it often has a high computational complexity in the model inference. Meanwhile, the drug-indication transition matrix of the probabilistic graph model usually needs to be marked by a professional doctor, or mined by means of human-computer cooperation (graph mining+artificial iterative evaluation). This kind of expert system-like model faces great challenges in the expansion of different hospitals and diverse patient situations. Therefore, a new method is needed to make breakthroughs in the field of intelligent drug recommendation.

By applying schemes of the present application, a drug recommendation may be made based on a first model according to related information of a target object. In addition, the first model is obtained by performing iterative processing on output information of a second model, and the role of the second model is to evaluate drug recommendation information. In this way, since an evaluation of drug recommendation information is introduced in the training of the first model, finally recommended drugs may be more accurate.

In addition, according to schemes provided by the present application, the first model is trained (or enhanced) by introducing an evaluation result indicating whether there is incompatibility between drugs in the iterative processing. Further, according to schemes provided by the present application, artificial intervention may be avoided as much as possible in the processing of model training and reinforcement learning, thereby ultimately ensuring that drugs recommended by a first model is an optimal solution that may avoid incompatibility.

According to an embodiment of the present application, a drug recommendation device is provided as shown in FIG. 6. The device includes:

an information acquisition module 61, configured to acquire related information of a target object;

a drug recommendation module 62, configured to determine drug recommendation information for the target object based on the related information of the target object and a first model, wherein the drug recommendation information contains information of at least one drug;

wherein the first model is a model obtained by performing iterative processing on output information of a second model, and the second model is used for evaluating drug recommendation information output by the first model during the iterative processing, to obtain an evaluation result of the drug recommendation information.

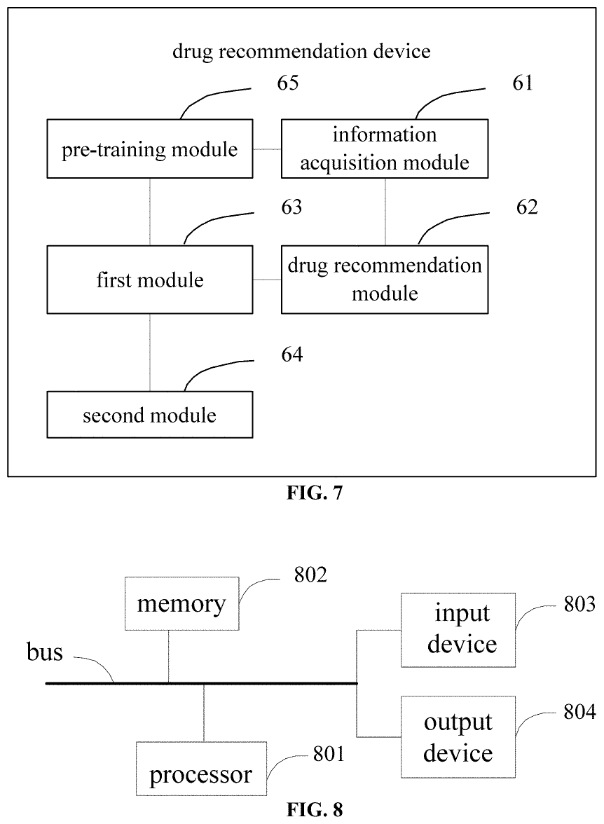

As shown in FIG. 7, the device further includes:

a first module 63, configured to obtain drug recommendation information for a patient, based on medical record information of the patient to be trained and a pre-trained first model; and determine whether training of the first model is completed based on an evaluation result; and

a second module 64, configured to obtain the evaluation result of the drug recommendation information for the patient based on the second model, wherein the evaluation result indicates whether there is incompatibility in the drug recommendation information for the patient.

The first module 63 is configured to update the training of the first model, to obtain an updated trained first model, in response to determining that the evaluation result indicates there is incompatibility in the drug recommendation information for the patient; re-obtain drug recommendation information for the patient by using the updated-trained first model and the medical record information of the patient to be trained; and determine that the training of the first model is not completed, until the evaluation result indicates that there is no incompatibility in the drug recommendation information for the patient.

The second module 64 is configured to update training of the second model, to obtain an updated-trained second model; and re-obtain an evaluation result of the drug recommendation information for the patient based on the updated-trained second model.

The second module 64 is configured to evaluate a drug combination output by the pre-trained first model based on the second model, to obtain a first reward value corresponding to the drug combination and a probability value of incompatibility between drugs in the drug combination, and take the first reward value and the probability value as the evaluation result.

The first module 63 is configured to obtain a second reward value corresponding to the drug recommendation information for the patient; determine a reward function result based on the second reward value and the evaluation result; and train the first model based on the reward function result, until the training of the first model is completed.

The device further includes:

a pre-training module 65, configured to acquire the medical record information of the patient to be trained and medical prescription data associated with the medical record information of the patient to be trained from a historical medical record database, wherein the medical prescription data contains data of at least one drug; perform vectorization processing on the medical record information of the patient to be trained and the medical prescription data, to obtain a medical record vector of the patient and at least one drug vector; and obtain the pre-trained first model based on the medical record vector of the patient and the at least one drug vector.

The drug recommendation module 62, is configured to perform word segmentation processing on the related information of the target object, to obtain related information after the word segmentation processing; perform vectorization processing on the related information after the word segmentation processing, to obtain vectorized related information; and determine the drug recommendation information for the target object based on the vectorized related information and the first model.

It should be pointed out here that the first module may send or save a first model into a drug recommendation module, after the training of the first model has been completed, so that the drug recommendation module may perform subsequent drug recommendation processing.

For functions of modules in drug recommendation devices according to embodiments of the present application, reference may be made to corresponding descriptions of the above method, and thus a detailed description thereof is omitted herein.

According to an embodiment of the present application, an electronic apparatus and a readable storage medium are provided in the present application.

As shown in FIG. 8, it is a block diagram showing an electronic apparatus applied with a drug recommendation method according to an embodiment of the present application. The electronic apparatus is intended to represent various forms of digital computers, such as laptop computers, desktop computers, workbenches, personal digital assistants, servers, blade servers, mainframe computers, and other suitable computers. The electronic apparatus may also represent various forms of mobile devices, such as personal digital processors, cellular phones, intelligent phones, wearable devices, and other similar computing devices. Components shown in the present application, their connections and relations, and their functions are merely examples, and are not intended to limit the implementation of the application described and/or required herein.

As shown in FIG. 8, the electronic apparatus includes: one or more processors 801, a memory 802, and interfaces for connecting various components, including a high-speed interface and a low-speed interface. The various components are interconnected using different buses and may be mounted on a common motherboard or otherwise installed as required. The processor may process instructions executed within the electronic apparatus, including instructions for storing in or on a memory, to display graphical information of a Graphical User Interface (GUI) on an external input/output device (such as a display device coupled to the interface). In other implementations, multiple processors and/or multiple buses may be used with multiple memories and multiple memories, if desired. Similarly, multiple electronic apparatuses may be connected, each apparatus providing some of the necessary operations (for example, as a server array, a group of blade servers, or a multiprocessor system). In FIG. 8, one processor 801 is shown as an example.

The memory 802 is a non-transitory computer-readable storage medium provided in the present application. The memory stores instructions executable by at least one processor, so that the at least one processor executes a drug recommendation method provided in the present application. The non-transitory computer-readable storage medium of the present application stores computer instructions, which are configured to enable a computer to execute a drug recommendation method provided in the present application.

As a non-transitory computer-readable storage medium, the memory 802 may be used to store non-transitory software programs, non-transitory computer executable programs, and modules, such as program instructions/modules corresponding to a drug recommendation method in embodiments of the present application (e.g., the information acquisition module, the drug recommendation module, the first module, the second module and the pre-training module shown in FIG. 7). The processor 801 executes various functional applications and data processing of the server by running non-transitory software programs, instructions, and modules stored in the memory 802, that is, to implement an above drug recommendation method in foregoing method embodiments.

The memory 802 may include a storage program area and a storage data area, where the storage program area may be used to store an application program required by an operating system or for at least one function; the storage data area may be used to store data created according to the use of an electronic apparatus. In addition, the memory 802 may include a high-speed random-access memory, and may also include a non-transitory memory, such as at least one magnetic disk storage device, a flash memory device, or other non-transitory solid-state storage device. In some embodiments, the memory 802 may optionally include a memory set remotely relative to the processor 801, and these remote memories may be connected to the electronic apparatus through a network. Examples of the above network include, but are not limited to, an Internet, an intranet, a local area network, a mobile communication network, and combinations thereof.

The electronic apparatus applied with a drug recommendation method may further include an input device 803 and an output device 804. The processor 801, the memory 802, the input device 803, and the output device 804 may be connected through a bus or in other manners. In FIG. 8, a connection through a bus is shown as an example.

The input device 803 may receive input numeric or character information, and generate key signal inputs related to a user setting and a function control of an electronic apparatus for analyzing a search result applied with a webpage rendering method, such as a touch screen, a keypad, a mouse, a trackpad, a touchpad, a pointing stick, one or more mouse buttons, a trackball, a joystick and other input devices. The output device 804 may include a display device, an auxiliary lighting device (for example, an LED), a haptic feedback device (for example, a vibration motor), and the like. The display device may include, but is not limited to, a Liquid Crystal Display (LCD), a Light Emitting Diode (LED) display, and a plasma display. In some implementations, the display device may be a touch screen.

Various implementations of the systems and technologies described herein may be implemented in a digital electronic circuit system, an integrated circuit system, an application specific integrated circuit (ASIC), a computer hardware, a firmware, a software, and/or combinations thereof. These various implementations may include: implementations in one or more computer programs, where the one or more computer programs are executable and/or interpretable on a programmable system including at least one programmable processor, programmable processor, where the programmable processor may be a dedicated or general-purpose programmable processor that may receive data and instructions from a storage system, at least one input device, and at least one output device, and may transmit the data and instructions to the storage system, the at least one input device, and the at least one output device.

These computing programs (also known as programs, software, software applications, or codes) include machine instructions of a programmable processor and may be implemented by using a high-level procedural and/or object-oriented programming language, and/or an assembly/machine language. As used herein, the terms “machine-readable medium” and “computer-readable medium” refer to any computer program product, apparatus, and/or device used to provide machine instructions and/or data to a programmable processor (for example, a magnetic disk, an optical disk, a memory, and a programmable logic device (PLD)), including machine-readable media that receives machine instructions as machine-readable signals. The term “machine-readable signal” refers to any signal used to provide machine instructions and/or data to a programmable processor.

In order to provide an interaction with a user, systems and techniques described herein may be implemented on a computer, where the computer includes: a display device (for example, a Cathode Ray Tube (CRT) or liquid crystal display (LCD) monitor) for displaying information to a user; and a keyboard and pointing device (such as a mouse or a trackball) through which a user may provide input to a computer. Other kinds of devices may also be used to provide interaction with a user. For example, a feedback provided to a user may be a sensory feedback in any form (for example, a visual feedback, an auditory feedback, or a haptic feedback), and a user input (including an acoustic input, a voice input, or a tactile input) may be received in any form.

The systems and technologies described herein may be implemented in a computing system including a background component (for example, as a data server), a computing system including a middleware component (for example, an application server), or a computing system including a front-end component (for example, a user computer with a graphical user interface or a web browser, through which the user may interact with an implementation of the systems and technologies described herein), or a computer system including any combination of such a background component, a middleware component, or a front-end component. The components of the system may be interconnected by any form or medium of digital data communication (such as, a communication network). Examples of a communication network include a Local Area Network (LAN), a Wide Area Network (WAN), and the Internet.

It should be understood the steps in the various processes described above may be reordered or omitted, or other steps may be added therein. For example, the steps described in the application may be performed parallelly, sequentially, or in different orders, as long as the desired results of the technical solutions disclosed in the application may be achieved, to which no limitation is made herein.

Embodiments of the present application relate to fields of artificial intelligence deep learning technology, intelligent recommendation, and knowledge graphs. According to the technical schemes of embodiments of the present application, a drug recommendation is made based on a first model according to related information of a target object. The first model is obtained by performing iterative processing on output information of a second model, where the role of the second model is to evaluate drug recommendation information. In this way, since the evaluation of drug recommendation information is introduced in training of the first model, so that finally recommended drugs may be more accurate.

It should be understood the steps in the various processes described above may be reordered or omitted, or other steps may be added therein. For example, the steps described in the application may be performed parallelly, sequentially, or in different orders, as long as the desired results of the technical solutions disclosed in the application may be achieved, to which no limitation is made herein.

The embodiments above do not constitute a limitation on the protection scope of the present application. It should be understood by those skilled in the art that various modifications, combinations, sub-combinations, and substitutions may be available according to design requirements and other factors. Any modifications, equivalent replacements and improvements made within the spirit and principle of the present application shall be covered within the protection scope of the present application.

What is claimed is:

1. A drug recommendation method, comprising:
acquiring related information of a target object; and
determining drug recommendation information for the target object based on the related information of the target object and a first model, wherein the drug recommendation information contains information of at least one drug,
wherein the first model is a model obtained by performing iterative processing on output information of a second model, and the second model is used for evaluating drug recommendation information output by the first model during the iterative processing, to obtain an evaluation result of the drug recommendation information.

2. The drug recommendation method according to claim 1, further comprising:
obtaining drug recommendation information for a patient, based on medical record information of the patient to be trained and a pre-trained first model;
obtaining an evaluation result of the drug recommendation information for the patient based on the second model, wherein the evaluation result indicates whether there is incompatibility in the drug recommendation information for the patient; and
determining whether training of the first model is completed based on the evaluation result.

3. The drug recommendation method according to claim 2, wherein the determining whether the training of the first model is completed based on the evaluation result comprises:
updating the training of the first model and updating training of the second model, to obtain an updated-trained first model and an updated-trained second model, in response to determining that the evaluation result indicates there is incompatibility in the drug recommendation information for the patient;
re-obtaining drug recommendation information for the patient by using the updated-trained first model and the medical record information of the patient to be trained, and re-obtaining an evaluation result of the drug recommendation information for the patient based on the updated-trained second model; and
determining that the training of the first model is not completed, until the evaluation result indicates that there is no incompatibility in the drug recommendation information for the patient.

4. The drug recommendation method according to claim 3, wherein the obtaining the evaluation result of the drug recommendation information for the patient based on the second model comprises:
evaluating a drug combination output by the pre-trained first model based on the second model, to obtain a first reward value corresponding to the drug combination and a probability value of incompatibility between drugs in the drug combination, and taking the first reward value and the probability value as the evaluation result.

5. The drug recommendation method according to claim 4, wherein the obtaining the drug recommendation information for the patient based on the medical record information of the patient to be trained and the pre-trained first model further comprises:
obtaining a second reward value corresponding to the drug recommendation information for the patient;
correspondingly, the method further comprises:
determining a reward function result based on the second reward value and the evaluation result; and
training the first model based on the reward function result, until the training of the first model is completed.

6. The drug recommendation method according to claim 2, further comprising:
acquiring the medical record information of the patient to be trained and medical prescription data associated with the medical record information of the patient to be trained from a historical medical record database, wherein the medical prescription data contains data of at least one drug;
performing vectorization processing on the medical record information of the patient to be trained and the medical prescription data, to obtain a medical record vector of the patient and at least one drug vector; and
obtaining the pre-trained first model based on the medical record vector of the patient and the at least one drug vector.

7. The drug recommendation method according to claim 1, wherein the determining the drug recommendation information for the target object based on the related information of the target object and the first model comprises:
performing word segmentation processing on the related information of the target object, to obtain related information after the word segmentation processing;
performing vectorization processing on the related information after the word segmentation processing, to obtain vectorized related information; and
determining the drug recommendation information for the target object based on the vectorized related information and the first model.

8. A drug recommendation device, comprising:
at least one processor; and
a memory communicatively connected to the at least one processor, wherein the memory stores instructions executable by the at least one processor, the instructions are executed by the at least one processor to enable the at least one processor to:
acquire related information of a target object; and
determine drug recommendation information for the target object based on the related information of the target object and a first model, wherein the drug recommendation information contains information of at least one drug,
wherein the first model is a model obtained by performing iterative processing on output information of a second model, and the second model is used for evaluating drug recommendation information output by the first model during the iterative processing, to obtain an evaluation result of the drug recommendation information.

9. The drug recommendation device according to claim 8, wherein the instructions are executed by the at least one processor to further enable the at least one processor to:
obtain drug recommendation information for a patient, based on medical record information of the patient to be trained and a pre-trained first model; and determine whether training of the first model is completed based on an evaluation result; and
obtain the evaluation result of the drug recommendation information for the patient based on the second model, wherein the evaluation result indicates whether there is incompatibility in the drug recommendation information for the patient.

10. The drug recommendation device according to claim 9, wherein the instructions are executed by the at least one processor to further enable the at least one processor to update the training of the first model, to obtain an updated-trained first model, in response to determining that the evaluation result indicates there is incompatibility in the drug recommendation information for the patient; re-obtain drug recommendation information for the patient by using the updated-trained first model and the medical record information of the patient to be trained; and determine that the training of the first model is not completed, until the evaluation result indicates that there is no incompatibility in the drug recommendation information for the patient; and
update training of the second model, to obtain an updated-trained second model;
and re-obtain an evaluation result of the drug recommendation information for the patient based on the updated-trained second model.

11. The drug recommendation device according to claim 10, wherein the instructions are executed by the at least one processor to further enable the at least one processor to evaluate a drug combination output by the pre-trained first model based on the second model, to obtain a first reward value corresponding to the drug combination and a probability value of incompatibility between drugs in the drug combination, and take the first reward value and the probability value as the evaluation result.

12. The drug recommendation device according to claim 11, wherein the instructions are executed by the at least one processor to further enable the at least one processor to obtain a second reward value corresponding to the drug recommendation information for the patient; determine a reward function result based on the second reward value and the evaluation result; and train the first model based on the reward function result, until the training of the first model is completed.

13. The drug recommendation device according to claim 9, wherein the instructions are executed by the at least one processor to further enable the at least one processor to:
acquire the medical record information of the patient to be trained and medical prescription data associated with the medical record information of the patient to be trained from a historical medical record database, wherein the medical prescription data contains data of at least one drug; perform vectorization processing on the medical record information of the patient to be trained and the medical prescription data, to obtain a medical record vector of the patient and at least one drug vector; and obtain the pre-trained first model based on the medical record vector of the patient and the at least one drug vector.

14. The drug recommendation device according to claim 8, wherein the instructions are executed by the at least one processor to further enable the at least one processor to perform word segmentation processing on the related information of the target object, to obtain related information after the word segmentation processing; perform vectorization processing on the related information after the word segmentation processing, to obtain vectorized related information; and determine the drug recommendation information for the target object based on the vectorized related information and the first model.

15. A non-transitory computer-readable storage medium for storing computer instructions, wherein the computer instructions, when executed by a computer, cause the computer to
acquire related information of a target object; and
determine drug recommendation information for the target object based on the related information of the target object and a first model, wherein the drug recommendation information contains information of at least one drug,
wherein the first model is a model obtained by performing iterative processing on output information of a second model, and the second model is used for evaluating drug recommendation information output by the first model during the iterative processing, to obtain an evaluation result of the drug recommendation information.

16. The non-transitory computer-readable storage medium according to claim 15, wherein the computer instructions, when executed by a computer, further cause the computer to:
obtain drug recommendation information for a patient, based on medical record information of the patient to be trained and a pre-trained first model;
obtain an evaluation result of the drug recommendation information for the patient based on the second model, wherein the evaluation result indicates whether there is incompatibility in the drug recommendation information for the patient; and
determine whether training of the first model is completed based on the evaluation result.

17. The non-transitory computer-readable storage medium according to claim 16, wherein the computer instructions, when executed by a computer, further cause the computer to:
update the training of the first model and update training of the second model, to obtain an updated-trained first model and an updated-trained second model, in response to determining that the evaluation result indicates there is incompatibility in the drug recommendation information for the patient;
re-obtain drug recommendation information for the patient by using the updated-trained first model and the medical record information of the patient to be trained, and re-obtain an evaluation result of the drug recommendation information for the patient based on the updated-trained second model; and
determine that the training of the first model is not completed, until the evaluation result indicates that there is no incompatibility in the drug recommendation information for the patient.

18. The non-transitory computer-readable storage medium according to claim 17, wherein the computer instructions, when executed by a computer, further cause the computer to:
evaluate a drug combination output by the pre-trained first model based on the second model, to obtain a first reward value corresponding to the drug combination and a probability value of incompatibility between drugs in the drug combination, and take the first reward value and the probability value as the evaluation result.

19. The non-transitory computer-readable storage medium according to claim 18, wherein the obtaining the drug recommendation information for the patient based on the medical record information of the patient to be trained and the pre-trained first model further comprises:
obtaining a second reward value corresponding to the drug recommendation information for the patient;
correspondingly, the computer instructions, when executed by a computer, further cause the computer to:
determine a reward function result based on the second reward value and the evaluation result; and
train the first model based on the reward function result, until the training of the first model is completed.

20. The non-transitory computer-readable storage medium according to claim 16, wherein the computer instructions, when executed by a computer, further cause the computer to:
acquire the medical record information of the patient to be trained and medical prescription data associated with the medical record information of the patient to be trained from a historical medical record database, wherein the medical prescription data contains data of at least one drug;
perform vectorization processing on the medical record information of the patient to be trained and the medical prescription data, to obtain a medical record vector of the patient and at least one drug vector; and
obtain the pre-trained first model based on the medical record vector of the patient and the at least one drug vector.
